(19) United States
(12) Patent Application Publication
(10) Pub. No.: US 2026/0341447 A1
(43) Pub. Date: Oct. 29, 2026

(54) A DIGITAL HEALTHCARE MOBILE APPLICATION FOR WARFARIN MANAGEMENT

(71) Applicants: Yu-chen HSIEH, San Pedro, CA (US); Julia E OLSEN, San Pedro, CA (US); SALVE THERAPEUTICS INC., San Pedro, CA (US); PRESIDENT AND FELLOWS OF HARVARD COLLEGE, Cambridge, MA (US); THE BRIGHAM AND WOMEN'S HOSPITAL, Boston, MA (US)

(72) Inventors: Yu-chen HSIEH, San Pedro, CA (US); Julia E OLSEN, San Pedro, CA (US); Stefan N LUKIANOV, San Pedro, CA (US); Shreya SANGAM, San Pedro, CA (US); Nancy ANORUO, San Pedro, CA (US)

(21) Appl. No.: 19/106,148

(22) PCT Filed: Aug. 24, 2023
(86) PCT No.: PCT/US2023/072814

§ 371 Date: Feb. 24, 2025

Related U.S. Application Data
(60) Provisional application No. 63/400,600, filed on Aug. 24, 2022.

(51) Int. Cl. G16H 20/10; G16H 50/30

(52) U.S. Cl. CPC G16H 20/10; G16H 50/30

(57) ABSTRACT

Systems and methods for monitoring an international normalized ratio (INR) of a patient are disclosed. In some embodiments, INR data associated with the patient is received from an INR level measurement device. In some embodiments, a warfarin dosage recommendation is received, and the warfarin dosage recommendation is based on the INR data. In some embodiments, one or more user interfaces are presented on a display, and the one or more user interfaces comprise a user interface element indicating the warfarin dosage recommendation.

### CROSS-REFERENCE TO RELATED APPLICATION

This application claims priority to and the benefit of U.S. Provisional Application No. 63/400,600 filed Aug. 24, 2022, the entire content of which is incorporated herein by reference in its entirety.

### TECHNICAL FIELD

This disclosure generally relates to systems for monitoring international normalized ratio (INR).

### BACKGROUND

Blood coagulation, which is also known as blood clotting, is a process in which the body solidifies blood to repair blood vessels or stop excessive bleeding. However, blood clotting can also lead to harmful clots that may block blood flow in vessels, which may cause different health problems. Anticoagulation clinics can help provide specialized care, such as with “blood thinning” and through the use of warfarin, an FDA-approved oral anticoagulant drug used to treat blood clots. For example, patients with atrial fibrillation (AFib) are sometimes prescribed warfarin to treat blood clots. Warfarin dosage adjustment may be complex due to its therapeutic range, and mismanagement may cause life-threatening events such as bleeding or clot formation. As a result, patients may have to undergo four blood draws a week initially, followed by one or more blood draws per month to perform INR tests, which may be time-consuming, inconvenient, and frustrating for the patients. Additionally, with increased influx of patients requiring prescriptions of warfarin and increased influx of health data, patients and doctors may require an easier way to communicate for more promptly address healthcare needs.

### SUMMARY

Systems and methods for monitoring an international normalized ratio (INR) of a patient are disclosed. In some embodiments, INR data associated with the patient is received from an INR level measurement device. In some embodiments, a warfarin dosage recommendation is received, and the warfarin dosage recommendation is based on the INR data. In some embodiments, one or more user interfaces are presented on a display, and the one or more user interfaces comprise a user interface element indicating the warfarin dosage recommendation.

In some embodiments, a method for monitoring INR level of a patient comprises, at a first device associated with the patient, receiving, from an INR level measurement device at a first location, first INR data associated with the INR level of the patient. In some embodiments, the method further comprises receiving, from the patient, an input indicating warfarin dosage associated with the patient, transmitting the first INR data and the warfarin dosage to a second device, and receiving a warfarin dosage recommendation from the second device. In some embodiments, the warfarin dosage recommendation is determined based on the transmitted first INR data and the transmitted warfarin dosage. In some embodiments, the method further comprises receiving second INR data associated with the INR level of the patient. In some embodiments, the second INR data is collected at a second location different the first location.

In some embodiments, the method further comprises presenting, on a display of the device associated with the patient, one or more user interfaces comprising: a first user interface element indicating the warfarin dosage recommendation, a second user interface element indicating a wellness of the INR level of the patient, the wellness of the INR level of the patient determined based on one or more of the first INR data received from the INR level measurement device at the first location and the second INR data collected at the second location, and a third user interface element indicating a trend of the INR level of the patient, the trend determined based on a composite analysis of the first INR data received from the INR level measurement device at the first location and the second INR data collected at the second location.

The embodiments disclosed above are only examples, and the scope of this disclosure is not limited to them. Particular embodiments may include all, some, or none of the components, elements, features, functions, operations, or steps of the embodiments disclosed above. Embodiments according to the invention are in particular disclosed in the attached claims directed to a method, a storage medium, a system and a computer program product, wherein any feature mentioned in one claim category, e.g., method, can be claimed in another claim category, e.g., system, as well. The dependencies or references back in the attached claims are chosen for formal reasons only. However, any subject matter resulting from a deliberate reference back to any previous claims (in particular multiple dependencies) can be claimed as well, so that any combination of claims and the features thereof are disclosed and can be claimed regardless of the dependencies chosen in the attached claims. The subject-matter which can be claimed comprises not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

### BRIEF DESCRIPTION OF THE DRAWINGS

FIG. 1 illustrates an exemplary system according to embodiments of this disclosure.

FIGS. 2A-2H illustrate exemplary user interfaces according to embodiments of this disclosure.

FIGS. 3A-3C illustrate an exemplary system according to embodiments of this disclosure.

FIG. 4 illustrates an exemplary system according to embodiments of this disclosure.

FIG. 5 illustrates an exemplary method according to embodiments of this disclosure.

FIG. 6 illustrates an example computer system according to embodiments of this disclosure.

### DESCRIPTION OF EXAMPLE EMBODIMENTS

In the following description of embodiments, reference is made to the accompanying drawings which form a part hereof, and in which it is shown by way of illustration specific embodiments which can be practiced. It is to be understood that other embodiments can be used, and structural changes can be made without departing from the scope of the disclosed embodiments.

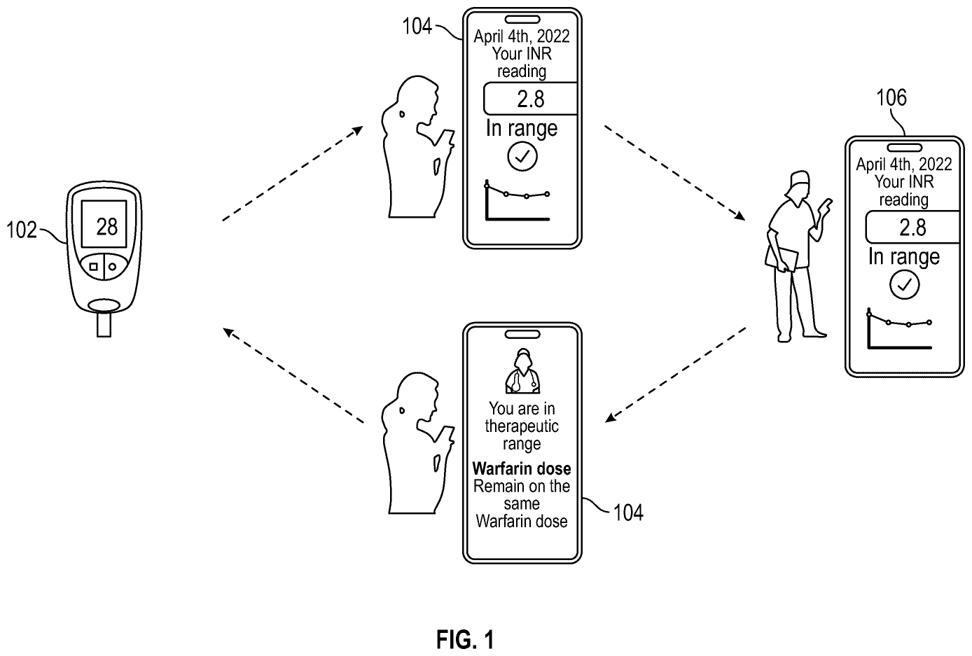

FIG. 1 illustrates an exemplary system 100 according to embodiments of this disclosure. In some embodiments, the system 100 comprises an INR level measurement device 102, a first device 104, and a second device 106. In some embodiments, the INR level measurement device 102 is a device configured to measure INR level of a patient. For example, the INR level measurement device 102 is a self-testing INR measurement machine, such as the Roche CoaguChek Vantus. In some embodiments, the INR level measurement device 102 is configured to communicate INR data to another device (e.g., first device 104, second device 106). The INR data may comprise the patient's INR level measured by the INR level measurement device. In some embodiments, the communication between the INR level measurement device 102 and this other device is wireless (e.g., via a Bluetooth connection). In some embodiments, the INR measurement device 102 is at a first location, and the first location may be a location away from a clinic (e.g., the patient's home, a caregiving facility). The INR data may comprise clotting data of the patient, and the patient's INR level may be determined from the clotting data (e.g., by a device of system 100).

As described in more detail herein, the communications between the INR level measurement device 102 and another device advantageously allows INR data to be communicated remotely, which would reduce trips to a clinic for collecting INR data (which indicates anticoagulation effects of warfarin) required to determine warfarin dosage for treating AFib. Additionally, this communication may advantageously allow more collection of INR data of a patient, which would improve a resolution of INR level monitoring that would facilitate more accurate determination of warfarin dosage for AFib treatment.

In some embodiments, the first device 104 is a device associated with a patient. For example, the device is the patient's mobile device. In some embodiments, the first device 104 is configured to install an application (e.g., a mobile application, software as a medical device) for performing the operations described herein. In some embodiments, the first device 104 is configured to present a web browser for performing the operations described herein. As described above, in some embodiments, the first device 104 is configured to receive INR data from the INR level measurement device 102, and this communication may be wireless (e.g., via a Bluetooth connection). In some embodiments, the first device 104 is configured to store the INR data received from the INR level measurement device 102.

In some embodiments, the first device 104 is configured to receive an input from the patient, and the input indicates a warfarin dosage associated with the patient. For example, the patient inputs his or her current warfarin dosage to the first device 104. As another example, the patient inputs his or her past warfarin dosage to the first device 104.

In some embodiments, the first device 104 is configured to transmit INR data (e.g., received from INR level measurement device 102) and warfarin dosage (e.g., received via the input described above). For example, as illustrated, the first device 104 transmits the INR data and the warfarin dosage to the second device 106, which may be a device associated with a clinician (e.g., cardiologists, pulmonologists, nurse practitioners, pharmacists) or clinic treating the patient (e.g., anticoagulation clinics). In some embodiments, the communications between the first device 104 and the second device 106 is facilitated via a server. It should be appreciated that transmission and receiving of data between two devices described herein may include direct transmission and receiving and indirect transmission and receiving (e.g., via a server, via a database, via another device such as a router, a bridge, a network controller, a mobile device, or a computer).

In some embodiments, the second device 106 is configured to receive INR data and warfarin dosage, which may be transmitted from the first device 104. In some embodiments, the INR data and warfarin dosage are received from a server storing data associate with the patient. In some embodiments, the second device 106 is configured to install an application (e.g., a mobile application, software as a medical device) for performing the operations described herein. In some embodiments, the second device 106 is configured to present a web browser for performing the operations described herein.

In some embodiments, the first device 104 is configured to receive a warfarin dosage recommendation (e.g., increase current dosage, decrease current dosage, a recommended dosage amount, a recommended frequency, a recommendation to visit the clinic). Warfarin, which is a blood thinner, may be used to reduce clotting. In some embodiments, a warfarin dosage recommendation comprising an increase dosage is determined in accordance with a determination that the clotting increased, and conversely, a warfarin dosage recommendation comprising a decrease dosage is determined in accordance with a determination that the clotting decreased.

For example, the first device 104 is configured to receive a warfarin dosage recommendation from the second device 106. As described in more detail below, the warfarin dosage recommendation may be communicated via a user interface presented on a display of the first device 104. The warfarin dosage recommendation may be communicated graphically and/or verbally on the user interface. In some embodiments, the warfarin dosage recommendation is additionally or alternatively communicated via a text message, a voice call, a video call, or any combination thereof.

In some embodiments, the warfarin dosage recommendation is determined based on the transmitted INR data and the transmitted warfarin dosage (from the first device 104). For example, the warfarin dosage recommendation is determined via an input to the second device 106 (e.g., from the clinician, from a personnel of the clinic) after the second device 106 receives the INR data and warfarin dosage transmitted from the first device 104 and presents the INR data and warfarin dosage for review. As another example, the warfarin dosage recommendation is determined by inputting the transmitted INR data and transmitted warfarin dosage to an algorithm (e.g., a clinical decision support algorithm) for determining an optimal warfarin dosage for the patient based on received data associated with the patient.

For instance, if the INR data indicates an improvement in the patient's INR level, then the warfarin dosage recommendation would comprise a dosage reduction from the received warfarin dosage. If the INR data indicates a worsening in the patient's INR level, then the warfarin dosage recommendation would comprise a dosage increase from the received warfarin dosage.

The system 100 advantageously allows patient data to be communicated more efficiently and more quickly, which results in more effective communications between patient and clinician (e.g., recommendations being determined more accurately and communicated more quickly). A quicker and more accurate determination would accelerate the patient's treatment and reduce a risk of administering a potentially incorrect warfarin dosage and other potential medical emergencies.

In some embodiments, the first device 104 is configured to receive INR data from a source other than the INR level measurement device 102. For example, the first device 104 is configured to receive INR data from the second device 106. This INR data from the second device 106 may be INR data collected at a second location, different from the location of the INR level measurement device 102 (e.g., a clinic treating the patient, a database storing the patient's INR data, a clinician or a clinic device).

In some embodiments, the first device 104 and/or the second device 106 comprise a display and is configured to present a user interface. For example, the user interface comprises an interface element indicating the warfarin dosage recommendation received by the first device 104, as described above and herein. As another example, the user interface comprises interface elements for presenting information to a user of the second device 106 (e.g., a clinician, a personnel of a clinic). As another example, the user interface comprises user interface elements for aggregating, filtering, and sorting patient data to facilitate a user (e.g., a clinician, a personnel of a clinic) reviewing data presented on the second device 106. The user interface may be a part of an application installed on a respective device. The user interface may be presented via a web browser of a respective device.

As another example, the user interface comprises a user interface element indicating a wellness of the INR level of the patient. For example, the wellness of the INR level is a classification (e.g., good, bad, medium, healthy, not healthy) indicating a condition of the patient's INR level. The wellness of the INR level of the patient may be determined based on the INR data received from the INR level measurement device 102, INR data collected from a location different from a location of the INR level measurement device 102, or both. For example, the patient's INR level is determined based on the INR data received from the INR level measurement device 102, INR data collected from a location different from a location of the INR level measurement device 102, or both, and the determined INR level is compared with one or more thresholds to determine the wellness of the INR level. In some embodiments, these thresholds are determined based on patient data (e.g., age, gender, other factors for determining a wellness associated with a level).

As another example, the user interface comprises a user interface element indicating a trend of the INR level of the patient. In some embodiments, the trend is determined based on a composite analysis of the first INR data received from the INR level measurement device at the first location and the second INR data collected at the second location. As an example, this user interface element comprises one or more charts, graphs, or visualizations showing the patent's INR levels. The patient's INR levels may be displayed concurrently with other data associated with patient, advantageously allowing the INR levels to be correlated with the other data and causes for improved/worsened INR levels to be more accurately determined.

In some embodiments, the user interface comprises a user interface element indicating a result of a combination of data from devices of system 100 (e.g., INR level measurement device 102, first device 104, second device 106). For example, the combined data comprises data from the patient and data from the clinic treating the patient. This user interface element may advantageously combine data from different sources and/or locations into formats that are more accessible to a user (e.g., a patient, a caregiver, a clinician, personnel at a clinic), allowing treatment decisions to be more effectively determined. In some embodiments, the user interface comprises a user interface element providing a link to educational information (e.g., for understanding the disease, treatment, etc.). Additional examples and details of the user interface are described below.

In some embodiments, the first device 104 is configured to receive data other than INR data and warfarin dosage (e.g., secondary data). This data may be further used to determine the warfarin dosage recommendation or other recommendations for facilitating patient treatment. For example, this data includes medical history of the patient, patient's family history, the patient's diet, the patient's psychosocial information, the patient's sleep information, the patient's activity information, data from the patient's health applications (e.g., fitness monitoring applications, sleep monitoring applications, vitals monitoring applications), metabolites information, prescription information, care information associated with the patient, feedback regarding current dosage recommendation, bruising information associated with the patient, bleeding information associated with the patient, biomarker information (e.g., coagulation factors, platelets), or any combination thereof. This data may be provided via an input from a user of the first device 104, received in response to an input from the user, or may be received automatically (e.g., the data is periodically synced).

In some embodiments, the first device 104 is configured to share data associated with a first application with a different application. For example, the first device 104 may share INR level monitoring information, warfarin dosage recommendation, or other patient data received via a first application with a social media application (e.g., for caregiving, for support) or a health-related application (e.g., for harmonizing treatment).

Although examples of the first device 104 is described with respect to a patient. It should be appreciated that the first device 104 may also be associated with someone else who would like to monitor the patient's INR level and review the patient's warfarin dosage recommendations (e.g., a caregiver).

In some embodiments, the second device 106 is configured to receive INR data, warfarin dosage adjustment, and visit information. These received data may be used to determine the warfarin dosage recommendation provided to the first device 104. The INR data may be data collected during patient visits. The warfarin dosage adjustment may be an input to a user of the second device 106 (e.g., a clinician, a personnel of the clinic) based on a review of data associated with the patient. Visit information may comprise data collected from the patient's visit (e.g., weight, height, vitals such as blood pressure, heart rate, and temperature, lab results) and recommendations provided to the patient during his or her visits. In addition to INR data, warfarin dosage adjustment, and visit information, the second device 106 may be configured to receive metabolites information, prescription/drug information, and care information associated with the patient.

In some embodiments, the first device 104 and/or the second device 106 are configured to receive patient data from another device. For example, the first device 104 and/or the second device 106 are configured to receive patient data from a virtual health record (VHR) from a server, and the received patient data may be used to determine warfarin dosage recommendation and perform analysis relating to INR level monitoring.

In some embodiments, the system 100 is configured to provide a projected or recommended target INR level. For example, based on past INR levels, the system 100

determines a target INR level at a time in the future (e.g., reach INR level =X at Y amount of time), which would encourage the patient to adhere to the treatment recommendations and to perform activities beneficial to treatment (e.g., exercise more, have a healthier diet). As an example, the target INR level is provided to a device of the system 100 (e.g., from a patient to first device 104, from a clinician to second device 106). As another example, the projected or recommended target INR level may be extrapolated from past INR levels, which may be advantageously collected by the first device 104 to allow a more accurate extrapolation. As another example, the target INR level may be determined via an algorithm based on data associated with the patient. As another example, the target INR level may be based on an input to a device of the system (e.g., second device 106) and transmitted to another device of the system (e.g., first device 104). As another example, the target INR level may be determined based on the patient's ability to meet previous target INR levels. If a patient has been able to meet previous target INR levels, then the determined target INR level may have a greater difference compared to previous target INR levels. If a patient has not been able to meet previous target INR levels, then the determined target INR level may have a smaller difference compared to previous target INR levels.

In some embodiments, one or more devices of the system 100 are configured to store and communicate clotting data. The clotting data may be communicated via encryption for security, and the data may be stored at a secured database. In some embodiments, one or more devices of the system 100 are configured to perform statistical analysis of patient data and/or data from clinicians. For example, a device may perform analysis of the data and present results of the analysis, such as trends, correlation, and averages, via the user interface.

In some embodiments, one or more devices of the system 100 are configured to present user notifications (e.g., via an alert of the device, via the user interface). For example, a device may determine whether a change in patient data (e.g., INR level, vitals) is greater than a threshold value, and in accordance with a determination that the change is greater than the threshold value, the device is configured to present the user notification.

In some embodiments, one or more devices of the system 100 are configured to determine calculations related to patient data. For example, a device is configured to calculate INR, PTT, hemoglobin, fibrillation, platelet activating factor, or any combination thereof. In some embodiments, one or more devices of the system 100 are configured to provide health status indicators. For example, a device is configured to perform a rule-based algorithm for classifying a measurement (e.g., a healthy measurement, an unhealthy measurement).

In some embodiments, one or more devices of system 100 are configured for encryption, access control, and audit trails (e.g., for HIPAA compliance and security). In some embodiments, one or more devices of system 100 are configured to perform algorithms for more efficient data processing, retrieval, and synchronization.

Although examples of systems herein are described with respect to receiving INR data and providing recommendations associated with warfarin usage, it should be appreciated that the disclosed systems may be configured to receive other kinds of data and provide other kinds of medical recommendations based on the received data.

Although examples of system 100 are described with respect to three devices, it should be appreciated that the system 100 may include less than or more than three devices. For example, there may be an additional device that performs operations described with respect to first device 104 (e.g., a caregiver's device, a device that provides additional inputs). As another example, there may be an additional device that performs operations described with respect to second device 106 (e.g., another clinician's device, a device that provides additional inputs). As another example, the system 100 may not comprise an INR level measurement device (e.g., INR levels collected outside a clinic may be inputted to first device 104).

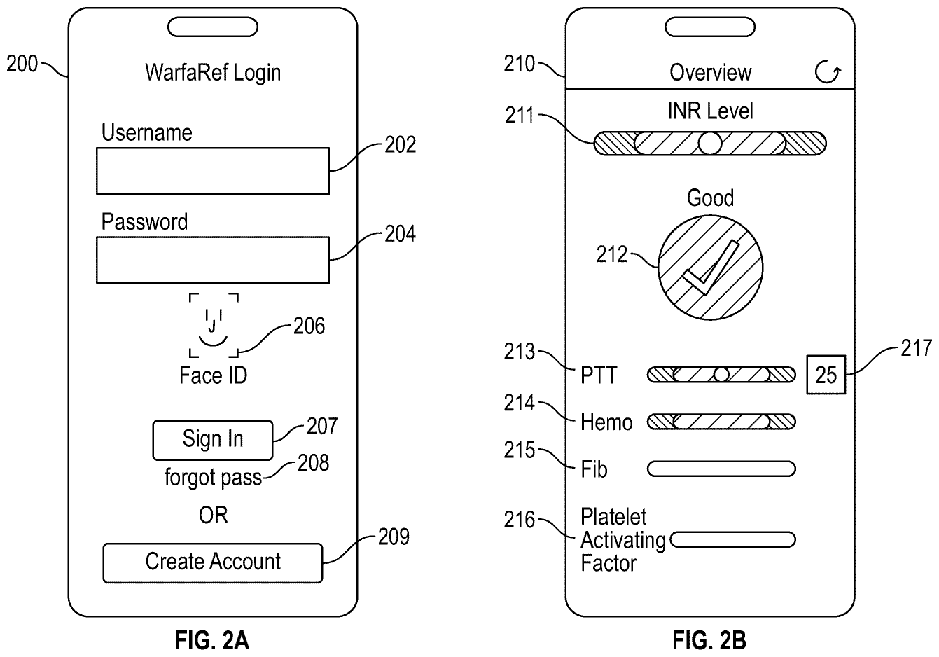

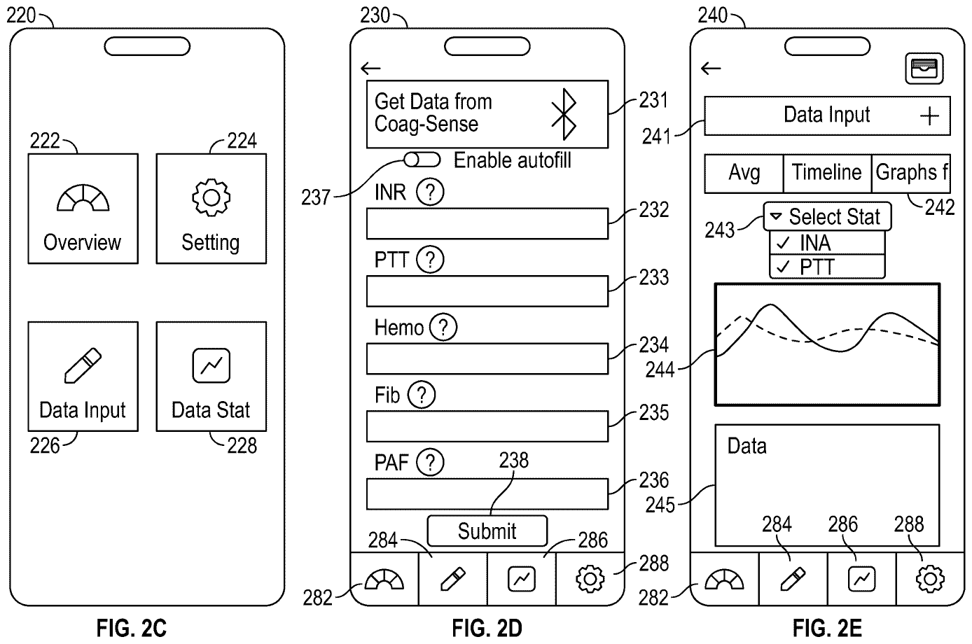

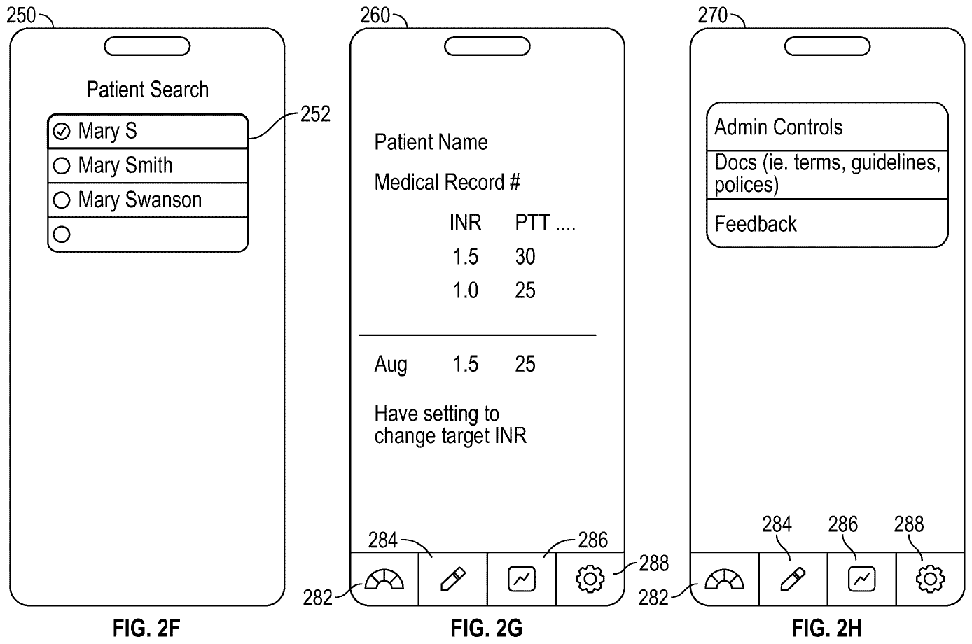

FIGS. 2A-2H illustrate exemplary user interfaces according to embodiments of this disclosure. These exemplary user interfaces may be presented by a device of system 100 (e.g., display of first device 104, display of second device 106). It should be appreciated that the user interfaces illustrated in FIGS. 2A-2H are exemplary, and that the user interfaces of system 100 may include fewer, different, or additional user interface elements than illustrated. For example, user interface elements illustrated from different figures may be combined onto a same user interface. As another example, fewer or additional user interface elements may appear on a user interface depending on the user (e.g., a patient user interface, a clinician user interface). It should also be appreciated that the navigations between user interfaces described with respect to FIGS. 2A-2E are exemplary.

FIG. 2A illustrates exemplary user interface 200. As illustrated, the user interface 200 comprises user interface elements for a logging in. For example, as illustrated, the user interface 200 comprises a user interface element 202 for receiving a username and a user interface element 204 for receiving a password. Also illustrated, the user interface 200 comprises a user interface element 206 for logging in via facial recognition. Also illustrated, the user interface 200 comprises a user interface element 207 for confirming input of login information, an accordance 208 for recovering/resetting password, and a user interface element 209 for creating an account. The login information allow system 100 to determine the type of user (e.g., patient, clinician, caregiver) logging in and provide appropriate user interfaces and user interface elements (e.g., the user has privileged associated with accessing the different user interfaces and user interface elements).

It should be appreciated that the login methods described with respect to user interface 200 are exemplary. A user may login via different methods than described. For example, a user may login via two-part authentication, an RSA token, or a clinic credential.

FIG. 2B illustrates exemplary user interface 210. In some embodiments, the user interface 210 comprises user interface elements associated with overview information. In some embodiments, after a user (e.g., patient, clinician, caregiver) logs in via the user interface 200, the device displays user interface 210. In some embodiments, the device displays user interface 210 after selection of an overview user interface element on e.g., user interface 220.

For example, as illustrated, the user interface 210 comprises a user interface element 211 indicating an INR level of a patient. As illustrated, the user interface element 211 comprises a bar comprising different regions corresponding to ranges of INR level and a marker indicating the INR level's value on the bar. In some embodiments, different ranges of levels have an associate color, which correspond to a wellness associated with a respective range (e.g., a range associated with “good” is green on a respective portion of the bar).

For example, the further left the marker is located on the user interface element 211, the higher the corresponding clotting level. Warfarin, which is a blood thinner, may be used to reduce clotting. In some embodiments, a warfarin dosage recommendation comprising an increase dosage is determined in accordance with a determination that the clotting increased (e.g., marker on user interface element 211 shifts towards the left), and conversely, a warfarin dosage recommendation comprising a decrease dosage is determined in accordance with a determination that the clotting decreased (e.g., marker on user interface element 211 shifts towards the right).

As illustrated, in some embodiments, the user interface 210 comprises a user interface element 212 indicating a wellness of the INR level of the patient. For example, the wellness of the INR level is a classification (e.g., good, as illustrated) indicating a condition of the patient's INR level. In some embodiments, the wellness of the INR level of the patient is determined based on one or more of the first INR data received from the INR level measurement device at the first location (e.g., INR level measurement device 102) and the second INR data collected at the second location (e.g., second device 106). For example, the patient's INR level is determined based on the INR data received from the INR level measurement device 102, INR data collected from a location different from a location of the INR level measurement device 102, or both, and the determined INR level is compared with one or more thresholds to determine the wellness of the INR level. In some embodiments, these thresholds are determined based on patient data (e.g., age, gender, other factors for determining a wellness associated with a level). The color of the user interface element 212 may also be associated with a wellness. For example, the user interface element 212 is green when the wellness is “good,” and the user interface element 212 is red when the wellness is “bad.”

As illustrated, the user interface 210 comprises user interface elements 213-216 indicating levels of other patent data. These user interface elements comprise bars comprising different regions corresponding to ranges of respective levels of patient data and a marker indicating a respective level's value on the bar. In some embodiments, different ranges of levels have an associate color, which correspond to a wellness associated with a respective range (e.g., a range associated with “good” is green on a respective portion of the bar). For example, as illustrated, the user interface element 213 indicates partial thromboplastin time (PTT) level, the user interface element 214 indicates hemoglobin level, the user interface element 215 indicates fibrillation, and the user interface element 216 indicates platelet activating factor.

In some embodiments, the user interface 210 comprises user interface element 217, and the user interface element 217 is next to one of user interface elements 213-216, as illustrated. The user interface element 217 may indicate a level of patient data associated with adjacent user interface element. For example, as illustrated, the user interface element 217 shows “25” (i.e., 25 seconds), which is a level of PTT. In some embodiments, the user interface element 217 may be in a color indicating a wellness of the corresponding level. For example, because 25 seconds is within a normal range of 25 to 30 seconds, the user interface element 217 is green.

In some embodiments, the user interface elements 211-217 are selectable, and a selection of a user interface element causes display of the user interface to update and present additional information associated with the selected user interface element. For example, if the user interface element 213 is selected, the user interface is updated to present a user interface element showing additional PTT level information.

It should be appreciated that user interface elements presenting information different from examples described with respect to FIG. 2B (e.g., other patient data (such as current warfarin dosage, warfarin dosage recommendation), as described with respect to FIG. 1) may be included on user interface 210. It should also be appreciated that the presented user interface elements may be updated based on an input to an associated device (e.g., user preference via user interface 270) or automatically to show user interface elements associated with data having the worse wellnesses.

FIG. 2C illustrates exemplary user interface 220. In some embodiments, the user interface 220 comprises user interface elements associated with menu items, and selection of the user interface elements would cause the device to update display to present a user interface corresponding to the selected user interface elements. In some embodiments, the user interface 220 comprises user interface elements 222-228. In this example, selection of user interface element 222 would cause the device to update display to present user interface 210. In this example, selection of user interface element 224 would cause the device to update display to present user interface 270. In this example, selection of user interface element 226 would cause the device to update display to present user interface 230. In this example, selection of user interface element 228 would cause the device to update display to present user interface 240.

FIG. 2D illustrates exemplary user interface 230. In some embodiments, the user interface 230 comprises user face elements for data input. In some embodiments, the device displays user interface 230 after selection of a data input user interface element (e.g., user interface 226) on user interface 220.

For example, as illustrated user interface element 231 is associated with a source of patient data that the device is configured to receive. For example, selection of user interface element 231 would cause the device (e.g., first device 104) to communicate with a device (e.g., INR level measurement device 102) to receive patient data (e.g., INR data), as described with respect to FIG. 1. As illustrated, the user interface element 231 indicates a pairing (e.g., a Bluetooth pairing) between an INR level measurement device and the device displaying the user interface 230. Selection of the user interface element 231 would cause the device to communicate with the paired INR level measurement device and receive INR data from the INR level measurement device.

In some embodiments, the user interface 230 comprises user interface element 237 adjacent to user interface element 231. In some embodiments, selection of user interface element 237 may enable or disable auto-filling (e.g., periodic automatic receiving of patient data). For example, as illustrated, user interface element 237 is adjacent to user interface element 231, and upon selection, is configured to cause enabling or disabling of auto-filling of received INR data (e.g., from INR level measurement device 102).

As illustrated, each of the user interface elements 232-236 is associated with receiving of a respective kind of patient data. Each of the user interface elements 232-236 may be updated to indicate respective patient data received by the device displaying user interface 230. For example, as illustrated, user interface element 232 is associated with INR level, user interface element 233 is associated with PTT, user interface element 234 is associated with hemoglobin level, user interface element 235 is associated with fibrillation, and user interface element 236 is associated with Paroxysmal Atrial Fibrillation (PAF).

Received data may be displayed on a respective user interface element. For example, after the device receives INR data from the INR level measurement device (e.g., after selection of user interface element 231), the user interface element 232 may show an INR level based on the received INR data. In some embodiments, the user interface comprises user interface element 238, and selection of the user interface element 238 causes received data indicated on the user interface 230 to be stored for further processing (e.g., for monitoring INR levels, for correlating INR levels with other patient data).

It should be appreciated that user interface elements associated with patient data different from examples described with respect to FIG. 2D (e.g., other patient data (such as current warfarin dosage, warfarin dosage recommendation), as described with respect to FIG. 1) may be included on user interface 230. It should also be appreciated that the presented user interface elements may be updated based on an input to an associated device (e.g., user preference via user interface 270) or automatically to show user interface elements associated with most frequently received patient data types.

FIG. 2E illustrates exemplary user interface 240. In some embodiments, the user interface 240 comprises user interface elements associated with data presentation. In some embodiments, the device displays user interface 240 after selection of a data presentation user interface element on user interface 220.

In some embodiments, selection of user interface element 241 causes the device to retrieve patient data for analysis or presentation on the user interface 240. For example, selection of user interface element 241 would cause the device to retrieve for example, INR data, data associated with user interface 240, or data described with respect to FIG. 1. The INR data being retrieved may be stored on the device, or selection of the user interface element 241 would cause the device to communicate with another device for receiving requested data.

In some embodiments, selection of user interface element 242 causes the device to perform and/or present a selected analysis of patient data. For example, as illustrated, a user may select between calculating an average, presenting timeline, or presenting one or more charts, graphs, or visualizations via the user interface element 242.

In some embodiments, the user interface element 243 allows a user to select available patient data to analyze and/or present on the user interface 240. For example, as illustrated, the user interface element 243 allows the user to select INR level, PTT, or both for display on user interface element 244, which may be graphs showing the two selected data, as illustrated. The horizontal axis and/or the vertical axis of the user interface element 244 may also be adjusted. For example, the horizontal axis is time, and axis may be adjusted to display the data over a selected time period.

In some embodiments, the patient's INR levels may be displayed concurrently with other data associated with patient on the user interface element 244, advantageously allowing the INR levels to be correlated with the other data and causes for improved/worsened INR levels to be more accurately determined.

In some embodiments, the user interface element 245 is configured to display more detailed data from specific points (e.g., time points) on the user interface element 244. The detailed data may be selected by a user or the device to view, for example, relevant numerical readings from the points corresponding to the selection. As an example, a user manually selects a location on the user interface element 244, and the detailed data corresponding to the selected location would display on user interface element 245.

In some embodiments, the user interface element 244 and/or user interface element 245 indicate a trend of patient data being analyzed (e.g., the INR level of the patient). For example, if the data being analyzed is the INR level of the patient, the trend is determined based on a composite analysis of the first INR data received from the INR level measurement device at the first location and the second INR data collected at the second location.

It should be appreciated that user interface elements associated with patient data (e.g., other patient data (such as current warfarin dosage, warfarin dosage recommendation), as described with respect to FIG. 1) and analyses (e.g., other statistical analyses, other correlation analyses, recommended reading analyses, date comparison analyses, other graphical analyses such as bar graphs and pie charts) different from examples described with respect to FIG. 2E may be included on user interface 240. For example, the user interface elements displayed on user interface 240 may be determined based on an attribute associated with the user. As an example, the user is a patient, and the user interface 240 is configured to display user interface elements that may be more useful to treatment of the patient. As another example, the user is a clinician, and the user interface 240 is configured to display user interface elements that may be more useful for the clinic to determine treatment for the patient.

FIG. 2F illustrates exemplary user interface 250. In some embodiments, the user interface 250 comprises user interface elements for searching. In some embodiments, the user interface 250 comprises user interface element 252, which may be an entry for searching for a patient. In some embodiments, the user interface 250 is accessible to clinician or personnel of a clinic, but not to another kind of user. As illustrated, in some embodiments, while a search term (e.g., patient name, patient ID, medical record number) is being provided to the user interface element 252, the user interface 250 may be updated to show suggestions, and selection of a suggestion would cause the device to update display to present data associated with the selected search result (e.g., on user interface 260).

FIG. 2G illustrates exemplary user interface 260. In some embodiments, the user interface 260 comprises user interface elements associated with search results. In some embodiments, the user interface 260 is display in response to a selection of a search result from user interface 250. In some embodiments, the user interface 260 is accessible to clinician or personnel of a clinic, but not to another kind of user.

As illustrated, for example, the user interface 260 comprises information associated with a search result, such as patient name, medical record number, INR levels (at different times), and PTTs (at different times). The averages of the INR levels and PTTs may also be shown on the user interface 260. It should be appreciated that other data and information associated with the search result may be presented on the user interface 260.

In some embodiments, the user interface 260 comprises a user interface element for updating or setting a target INR level for the patient associated with the search result. The target INR level may be determined and updated as described with respect to FIG. 1. In some embodiments, in accordance with a determination that the target INR level is updated (e.g., at second device 106), the target INR level is communicated to another device (e.g., to first device 104).

FIG. 2H illustrates an exemplary user interface 270. In some embodiments, the user interface 270 comprises user interface elements associated with settings. In some embodiments, the device displays user interface 270 after selection of a settings user interface element on user interface 220. In some embodiments, the user interface 270 comprises user interface elements for setting or updating administrative controls, accessing documents (e.g., terms, guidelines, policies), and providing feedback (e.g., to the developer, to a clinician, to a clinic). In some embodiments, the user interface 270 comprises user interface elements for configuring other user interfaces, as described above.

In some embodiments, the user interface includes user interface elements for navigating (e.g., causing the device to update from a first user interface to a second user interface) to other user interfaces. For example, the user interfaces 230, 240, 260, and 270 comprise user interface element 282 for navigating to user interface 210, user interface element 284 for navigating to user interface 230, user interface element 286 for navigating to user interface 240, and user interface element 288 for navigating to user interface 270.

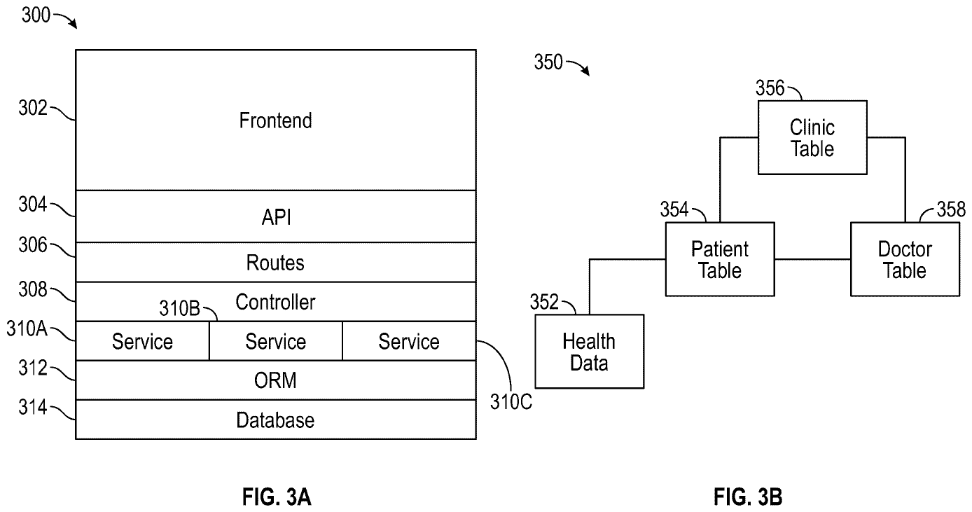

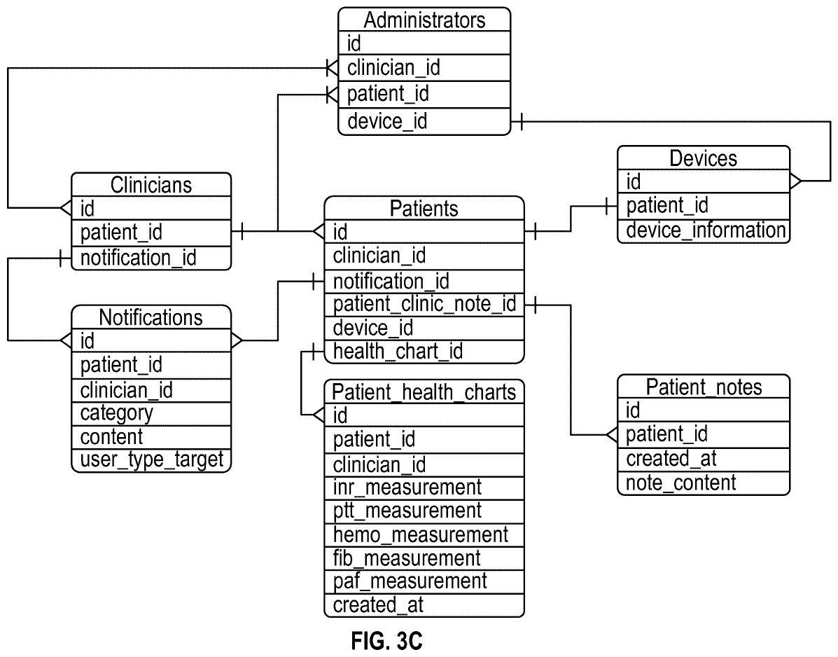

FIG. 3A-3C illustrate an exemplary system according to embodiments of this disclosure. In some embodiments, the system 100 comprises system 300, and devices of system 100 (e.g., first device 104, second device 106) are configured to communicate with components of system 300. For example, FIG. 3A shows data retrieval via a user interface (front end) through application programming interface (API) to secure patient data storage server; FIG. 3B shows patient data distribution between separate application user groups; FIG. 3C shows in-application interactions between users (e.g., patients, clinicians), application administrators, and external devices (e.g., INR level measurement device, home test kits, EHRs).

As illustrated, the system 300 comprises front end 302, API 304, routes 306, controller 308, services 310A-310C, and database 314. In some embodiments, the front end 302 is associated with a device of a user of system 100 (e.g., first device 104, second device 106).

The API 304 is configured for communication between the front end 302 and backend (e.g., server, database 314). In some embodiments, the routes 306 comprise routes for data transfers.

In some embodiments, the controller 308 is configured for server-side responses (e.g., database 314). In some embodiments, the server is object-related mapped (ORM) for web application features, which may allow a device to interface with a HIPAA-compliant secure cloud storage and/or EHRs to retrieve patient data. In some embodiments, the services 310A-310C are configured to return information associated with controller 308. In some embodiments, the database 314 is configured to store patient data, allowing the data to be accessible for clinics, clinicians, caregivers, and associate devices to determine treatment recommendations and monitor patient data.

FIG. 3B illustrate exemplary data 350 that may be used by system 100. In some embodiments, the data 350 comprise patient data described herein. In some embodiments, the data 350 comprises health data 352, patient table 354, clinic table 356, and doctor table 358.

In some embodiments, a disclosed system (e.g., system 100, system 300) is associated with one or more entities. For example, the entities comprise administrators, clinicians, patients, devices, notifications, patient health charts, and patient notes. FIG. 3C illustrates exemplary relationships between the different entities.

As illustrated, one or more administrators are associated with IDs and are related to clinicians, patients, and devices (e.g., devices associated with the clinicians and patients, other devices used to perform the operations described herein), which have their respective IDs. One or more clinicians are associated with IDs and are related to the one or more patients and one or more notifications, which have their respective IDs. One or more patients are associated with IDs and are related to one or more clinicians, one or more notifications, one or more patient health charts, one or more patient notes, and one or more devices, which have their respective IDs.

One or more devices are associated with IDs and respective device information and are related to one or more administrators. One or more notifications are associated with IDs, a category, content, and target, and are related to one or more clinicians and one or more patients. One or more patient health cares are associated with IDs and information related to patient ID, associated clinic ID, INR level, PTT, hemoglobin level, fibrillation measurement, PAF measurement, other patient data, and creation time. In some embodiments, the one or more patient health charts correspond to health data 352, patient table 354, or both. One or more patient notes are associated with IDs, creation time, and content (e.g., notes and information associated with a corresponding patient), and are related to one or more patients.

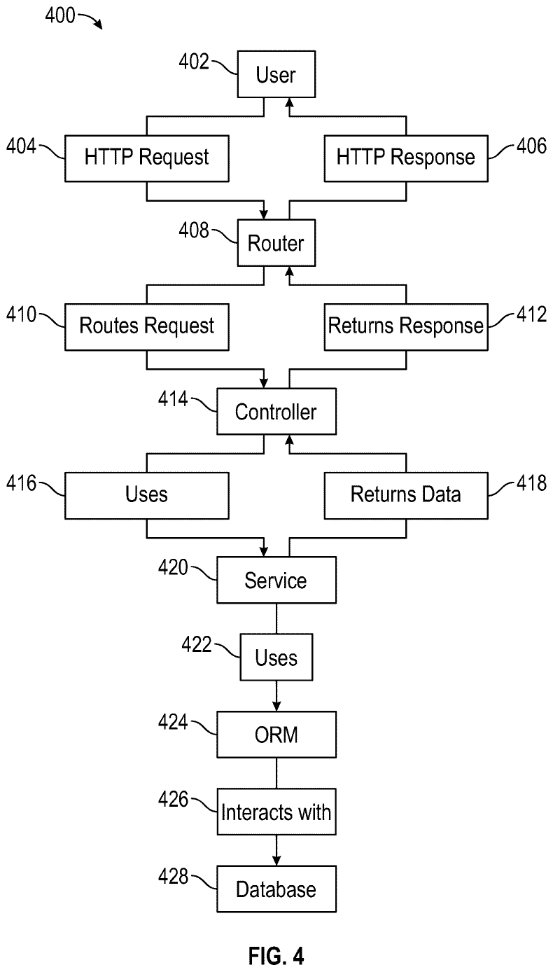

FIG. 4 illustrates an exemplary system according to embodiments of this disclosure. FIG. 4 illustrates an example interaction 400 between components of a disclosed system (system 300). For example, FIG. 4 shows an extension of FIG. 3A demonstrating the bidirectional deposit and retrieval of patient data by a user via a disclosed mobile application's web pathways. In some embodiments, the interaction 400 is an example of a website application (for performing the operations described herein) that stores and returns data. For example, as illustrated, a user 402 (e.g., clinician, patient) may transmit data to a router 408 via an HTTP request 404 and receive data from the router 408 via an HTTP response 406 (e.g., part of routes 306). The router 408 may route the request (410) to controller 414 (e.g., controller 308) and receive a response (412) from the controller 414.

The controller 414 may use (416) services 420 (e.g., services 310A-310C) and the services 420 may return data (418) to the controller 414. The service 420 may use (422) ORM 424 (e.g., ORM 312), which may interact with (426) database 428 (e.g., database 314).

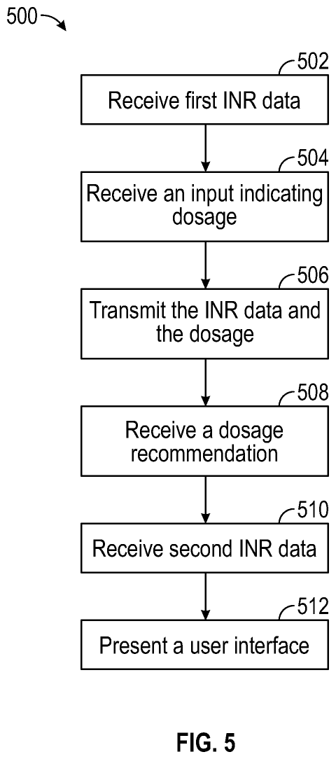

FIG. 5 illustrates an exemplary method 500 according to embodiments of this disclosure. In some embodiments, the steps of method 500 are performed by one or more components of system 100 and/or system 300. For example, steps of method 500 are performed by first device 104, second device 106, a stakeholder device, a server, or any combination thereof. It should be appreciated that steps described with respect to FIG. 5 are exemplary.

The method 500 may include fewer steps, additional steps, or different order of steps than described. It is appreciated that the steps of method 500 leverage the features and advantages of the disclosed systems (e.g., system 100, system 300).

In some embodiments, the method 500 comprises receiving, from an INR level measurement device at a first location, first INR data associated with the INR level of the patient (step 502). For example, as described with respect to an example of FIG. 1, first device 104 receives INR data associated with an INR level of a patient from INR level measurement device 102 at a location away from a clinic (e.g., home of patient). In some embodiments, the first INR data is received via a wireless connection (e.g., via a Bluetooth connection). In some embodiments, the first INR data comprises clotting data of the patient, and the INR level of the patient is determined based on the clotting data of the patient.

In some embodiments, the method 500 comprises receiving, from the patient, an input indicating warfarin dosage associated with the patient (step 504). For example, as described with respect to an example of FIG. 1, the first device 104 receives an input indicating patient's warfarin dosage via user interfaces 200-270.

In some embodiments, the method 500 comprises transmitting the first INR data and the warfarin dosage to a second device (step 506). For example, as described with respect to an example of FIG. 1, the first device 104 transmits the received INR data (e.g., measured from INR level measurement device 102) and the received warfarin dosage to second device 106.

In some embodiments, the method 500 comprises receiving a warfarin dosage recommendation from the second device (step 508). For example, as described with respect to an example of FIG. 1, the first device 104 receives a warfarin dosage recommendation from the second device 106. In some embodiments, the warfarin dosage recommendation is determined based on the transmitted first INR data and the transmitted warfarin dosage. For example, as described with respect to an example of FIG. 1, the warfarin dosage recommendation received by the first device 104 is based on the INR data and warfarin dosage transmitted by the first device 104. As an example, an input associated with the warfarin dosage recommendation is provided to second device 106 after analyzing the INR data and warfarin dosage received from the first device 104. In some embodiments, the warfarin dosage recommendation is determined by the second device 106 (e.g., clinician's device) or another device in communication with second device 106 (e.g., a server configured to determine the warfarin dosage recommendation based on the received data and transmit the recommendation to first device 104 and/or second device 106).

In some embodiments, the warfarin dosage recommendation is determined based on an input provided to the second device in response to the transmission of the first INR data and the warfarin dosage. For example, the warfarin dosage recommendation is determined based on a clinician's input to the second device in response to reviewing the first INR data and the warfarin dosage, as described with respect to FIG. 1.

In some embodiments, the warfarin dosage recommendation is determined by the second device. For example, the warfarin dosage recommendation is determined by the second device (e.g., a clinician's device, a server) based on the first INR data and the warfarin dosage received by the second device, as described with respect to FIG. 1.

In some embodiments, inputting the first INR data and the warfarin dosage into a machine learning model. For example, the second device comprises the machine learning model, and in response to receiving the first INR data and the warfarin dosage, the machine learning model is configured to determine and output a warfarin dosage recommendation.

In some embodiments, the warfarin dosage recommendation is received by a third device, different from the first device and the second device. For example, the warfarin dosage recommendation is received by a device different from first device 104 and second device 106, such as a caregiver's device or a clinic personnel's device.

In some embodiments, the method 500 comprises receiving second INR data associated with the INR level of the patient, wherein the second INR data is collected at a second location different the first location (step 510). For example, as described with respect to an example of FIG. 1, the first device 104 receives INR data other than data received from the INR level measurement device 102, for example, INR level collected at a clinic.

In some embodiments, the method 500 comprises presenting, on a display of the device associated with the patient one or more user interfaces (step 512). For example, as described with respect to examples of FIG. 1 and 2A-2H, the first device 104 is configured to present one or more user interfaces disclosed herein.

In some embodiments, the one or more user interfaces comprises a first user interface element indicating the warfarin dosage recommendation. For example, as described with respect to examples of FIG. 1 and 2A-2H, the first device 104 is configured to present a user interface element that indicates a warfarin dosage recommendation (e.g., display a clinician's warfarin dosage recommendation on a patient's device, display a warfarin dosage recommendation determined by a device of system 100 on a patient's device).

In some embodiments, the one or more user interfaces comprise a user interface element indicating a wellness of the INR level of the patient. For example, as described with respect to an example of FIG. 2B, the first device 104 is configured to present a user interface element that indicates a wellness of the patient's INR level (e.g., user interface element 212).

In some embodiments, the wellness of the INR level of the patient is determined based on one or more of the first INR data received from the INR level measurement device at the first location and the second INR data collected at the second location. For example, as described with respect to examples of FIG. 1 and 2A-2H, the wellness of the INR level is determined based on measurements collected from the INR level measurement device 102, measurements collected at a clinic, or both.

In some embodiments, the one or more user interfaces comprise a user interface element indicating a trend of the INR level of the patient. For example, as described with respect to an example of FIG. 2E, the first device 104 is configured to present the user interface element 240 that shows a trend of the patient's INR level (e.g., user interface element 244).

In some embodiments, the trend is determined based on a composite analysis of the first INR data received from the INR level measurement device at the first location and the second INR data collected at the second location. For example, as described with respect to an example of FIG. 2E, the trend indicated by user interface element 244 is determined based on a composite analysis of data including INR data collected from INR level measurement device 104 and INR data collected at a clinic.

In some embodiments, the third user interface element further indicates a correlation between the INR level and secondary data. For example, as described with respect to FIGS. 1 and 2A-2H, the user interface element 244 can indicate a correlation between the INR level and secondary data described with respect to FIG. 1 (e.g., for determining a cause for the INR level).

In some embodiments, the method 500 further comprises further comprising receiving secondary data. For example, one or more devices of system 100 are configured to receive secondary data, as described with respect to FIG. 1. In some embodiments, the secondary data comprises Virtual Health Record (VHR). In some embodiments, the warfarin dosage recommendation is determined further based on the secondary data.

In some embodiments, the method 500 further comprises in accordance with a determination that the INR level of the patient is below a target INR level, presenting, on the display, a first user interface element indicating that the INR level is below the target INR level; and in accordance with a determination that the INR level of the patient is not below the target INR level, presenting, on the display, a second user interface element indicating that the INR level is not below the target INR level. For example, as described with respect to FIGS. 1 and 2A-2H, one or more devices of system 100 are configured to present user interface elements indicating whether the INR level exceed a target INR level.

In some embodiments, the method 500 further comprises presenting, on the display, a user interface element indicating a target INR level and a target time for reaching the target INR level, wherein the target INR level and the target time are determined based on one or more of the INR level of the patient, the warfarin dosage, and the warfarin dosage recommendation. For example, as described with respect to FIGS. 1 and 2A-2H, one or more devices of system 100 are configured to present user interface elements indicating target INR level and a target time for reaching the target INR level.

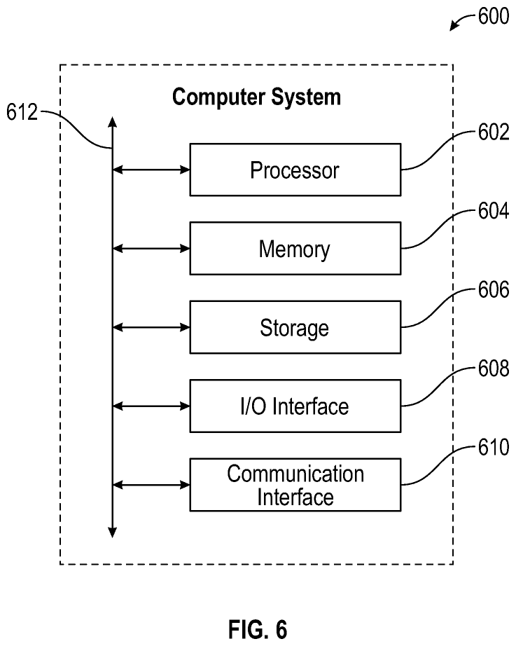

FIG. 6 illustrates an example computer system 600. In particular embodiments, one or more computer systems 600 perform one or more steps of one or more methods described or illustrated herein. In particular embodiments, one or more computer systems 600 provide functionality described or illustrated herein. In particular embodiments, software running on one or more computer systems 600 performs one or more steps of one or more methods described or illustrated herein or provides functionality described or illustrated herein. Particular embodiments include one or more portions of one or more computer systems 600. Herein, reference to a computer system may encompass a computing device, and vice versa, where appropriate. Moreover, reference to a computer system may encompass one or more computer systems, where appropriate. For example, first device 104 and second device 106 comprise computer system 600.

This disclosure contemplates any suitable number of computer systems 600. This disclosure contemplates computer system 600 taking any suitable physical form. As example and not by way of limitation, computer system 600 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 600 may include one or more computer systems 600; be unitary or distributed; span multiple locations; span multiple machines; span multiple data centers; or reside in a cloud, which may include one or more cloud components in one or more networks. Where appropriate, one or more computer systems 600 may perform without substantial spatial or temporal limitation one or more steps of one or more methods described or illustrated herein. As an example and not by way of limitation, one or more computer systems 600 may perform in real time or in batch mode one or more steps of one or more methods described or illustrated herein. One or more computer systems 600 may perform at different times or at different locations one or more steps of one or more methods described or illustrated herein, where appropriate.

In particular embodiments, computer system 600 includes a processor 602, memory 604, storage 606, an input/output (I/O) interface 608, a communication interface 610, and a bus 612. Although this disclosure describes and illustrates a particular computer system having a particular number of particular components in a particular arrangement, this disclosure contemplates any suitable computer system having any suitable number of any suitable components in any suitable arrangement.

In particular embodiments, processor 602 includes hardware for executing instructions, such as those making up a computer program. As an example and not by way of limitation, to execute instructions, processor 602 may retrieve (or fetch) the instructions from an internal register, an internal cache, memory 604, or storage 606; decode and execute them; and then write one or more results to an internal register, an internal cache, memory 604, or storage 606. In particular embodiments, processor 602 may include one or more internal caches for data, instructions, or addresses. This disclosure contemplates processor 602 including any suitable number of any suitable internal caches, where appropriate. As an example and not by way of limitation, processor 602 may include one or more instruction caches, one or more data caches, and one or more translation lookaside buffers (TLBs). Instructions in the instruction caches may be copies of instructions in memory 604 or storage 606, and the instruction caches may speed up retrieval of those instructions by processor 602. Data in the data caches may be copies of data in memory 604 or storage 606 for instructions executing at processor 602 to operate on; the results of previous instructions executed at processor 602 for access by subsequent instructions executing at processor 602 or for writing to memory 604 or storage 606; or other suitable data.

The data caches may speed up read or write operations by processor 602. The TLBs may speed up virtual-address translation for processor 602. In particular embodiments, processor 602 may include one or more internal registers for data, instructions, or addresses. This disclosure contemplates processor 602 including any suitable number of any suitable internal registers, where appropriate. Where appropriate, processor 602 may include one or more arithmetic logic units (ALUs); be a multi-core processor; or include one or more processors 602. Although this disclosure describes and illustrates a particular processor, this disclosure contemplates any suitable processor.

In particular embodiments, memory 604 includes main memory for storing instructions for processor 602 to execute or data for processor 602 to operate on. As an example and not by way of limitation, computer system 600 may load instructions from storage 606 or another source (such as, for example, another computer system 600) to memory 604. Processor 602 may then load the instructions from memory 604 to an internal register or internal cache. To execute the instructions, processor 602 may retrieve the instructions from the internal register or internal cache and decode them. During or after execution of the instructions, processor 602 may write one or more results (which may be intermediate or final results) to the internal register or internal cache. Processor 602 may then write one or more of those results to memory 604. In particular embodiments, processor 602 executes only instructions in one or more internal registers or internal caches or in memory 604 (as opposed to storage 606 or elsewhere) and operates only on data in one or more internal registers or internal caches or in memory 604 (as opposed to storage 606 or elsewhere). One or more memory buses (which may each include an address bus and a data bus) may couple processor 602 to memory 604. Bus 612 may include one or more memory buses, as described below. In particular embodiments, one or more memory management units (MMUs) reside between processor 602 and memory 604 and facilitate accesses to memory 604 requested by processor 602. In particular embodiments, memory 604 includes random access memory (RAM). This RAM may be volatile memory, where appropriate, this RAM may be dynamic RAM (DRAM) or static RAM (SRAM). Moreover, where appropriate, this RAM may be single-ported or multi-ported RAM. This disclosure contemplates any suitable RAM. Memory 604 may include one or more memories 604, where appropriate. Although this disclosure describes and illustrates particular memory, this disclosure contemplates any suitable memory.

In particular embodiments, storage 606 includes mass storage for data or instructions. As an example and not by way of limitation, storage 606 may include a hard disk drive (HDD), a floppy disk drive, flash memory, an optical disc, a magneto-optical disc, magnetic tape, or a Universal Serial Bus (USB) drive or a combination of two or more of these. Storage 606 may include removable or non-removable (or fixed) media, where appropriate. Storage 606 may be internal or external to computer system 600, where appropriate. In particular embodiments, storage 606 is non-volatile, solid-state memory. In particular embodiments, storage 606 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 606 taking any suitable physical form. Storage 606 may include one or more storage control units facilitating communication between processor 602 and storage 606, where appropriate. Where appropriate, storage 606 may include one or more storages 606. Although this disclosure describes and illustrates particular storage, this disclosure contemplates any suitable storage.

In particular embodiments, I/O interface 608 includes hardware, software, or both, providing one or more interfaces for communication between computer system 600 and one or more I/O devices. Computer system 600 may include one or more of these I/O devices, where appropriate. One or more of these I/O devices may enable communication between a person and computer system 600. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 608 for them. Where appropriate, I/O interface 608 may include one or more device or software drivers enabling processor 602 to drive one or more of these I/O devices. I/O interface 608 may include one or more I/O interfaces 608, where appropriate. Although this disclosure describes and illustrates a particular I/O interface, this disclosure contemplates any suitable I/O interface.

In particular embodiments, communication interface 610 includes hardware, software, or both providing one or more interfaces for communication (such as, for example, packet-based communication) between computer system 600 and one or more other computer systems 600 or one or more networks. As an example and not by way of limitation, communication interface 610 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 610 for it. As an example and not by way of limitation, computer system 600 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 600 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 600 may include any suitable communication interface 610 for any of these networks, where appropriate. Communication interface 610 may include one or more communication interfaces 610, where appropriate.

Although this disclosure describes and illustrates a particular communication interface, this disclosure contemplates any suitable communication interface.

In particular embodiments, bus 612 includes hardware, software, or both coupling components of computer system 600 to each other. As an example and not by way of limitation, bus 612 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 612 may include one or more buses 612, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

In some embodiments, a non-transitory computer readable storage medium stores one or more programs, and the one or more programs includes instructions. When the instructions are executed by an electronic device (e.g., system 100, system 300, system 600) with one or more processors and memory, the instructions cause the electronic device to perform the methods described with respect to FIGS. 1-5.

In some embodiments, a method for monitoring INR level of a patient comprises: at a first device associated with the patient: receiving, from an INR level measurement device at a first location, first INR data associated with the INR level of the patient; receiving, from the patient, an input indicating warfarin dosage associated with the patient; transmitting the first INR data and the warfarin dosage to a second device; receiving a warfarin dosage recommendation from the second device, wherein the warfarin dosage recommendation is determined based on the transmitted first INR data and the transmitted warfarin dosage; receiving second INR data associated with the INR level of the patient, wherein the second INR data is collected at a second location different the first location; and presenting, on a display of the device associated with the patient, one or more user interfaces comprising: a first user interface element indicating the warfarin dosage recommendation, a second user interface element indicating a wellness of the INR level of the patient, the wellness of the INR level of the patient determined based on one or more of the first INR data received from the INR level measurement device at the first location and the second INR data collected at the second location, and a third user interface element indicating a trend of the INR level of the patient, the trend determined based on a composite analysis of the first INR data received from the INR level measurement device at the first location and the second INR data collected at the second location.

In some embodiments, the method further comprises receiving secondary data, wherein the warfarin dosage recommendation is determined further based on the secondary data.

In some embodiments, the secondary data comprises Virtual Health Record (VHR).

In some embodiments, the method further comprises: in accordance with a determination that the INR level of the patient is below a target INR level, presenting, on the display, a first user interface element indicating that the INR level is below the target INR level; and in accordance with a determination that the INR level of the patient is not below the target INR level, presenting, on the display, a second user interface element indicating that the INR level is not below the target INR level.

In some embodiments, the method further comprises presenting, on the display, a user interface element indicating a target INR level and a target time for reaching the target INR level, wherein the target INR level and the target time are determined based on one or more of the INR level of the patient, the warfarin dosage, and the warfarin dosage recommendation.

In some embodiments, the first INR data is received via a wireless connection.

In some embodiments, the warfarin dosage recommendation is received by a third device, different from the first device and the second device.

In some embodiments, the first INR data comprises clotting data of the patient, and the INR level of the patient is determined based on the clotting data of the patient.

In some embodiments, the method further comprises inputting the first INR data and the warfarin dosage into a machine learning model.

In some embodiments, the warfarin dosage recommendation is determined based on an input provided to the second device in response to the transmission of the first INR data and the warfarin dosage.

In some embodiments, the warfarin dosage recommendation is determined by the second device.

In some embodiments, the third user interface element further indicates a correlation between the INR level and secondary data.

In some embodiments, a system comprises: an INR level measurement device at a first location, a first device comprising a display, a second device, and one or more processors, wherein the one or more processors are configured to perform any of the above methods.

In some embodiments, a non-transitory computer-readable medium stores instructions that, when executed by one or more processors, cause the one or more processors to perform any of above methods.

Those skilled in the art will recognize that the systems described herein are representative, and deviations from the explicilty disclosed embodiments are within the scope of the disclosure. For example, some embodiments include additional sensors or cameras, such as cameras covering other parts of the electromagnetic spectrum, can be devised using the same principles.

Although the disclosed embodiments have been fully described with reference to the accompanying drawings, it is to be noted that various changes and modifications will become apparent to those skilled in the art. Such changes and modifications are to be understood as being included within the scope of the disclosed embodiments as defined by the appended claims.

The terminology used in the description of the various described embodiments herein is for the purpose of describing particular embodiments only and is not intended to be limiting.

As used in the description of the various described embodiments and the appended claims, the singular forms “a”, “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

1. A method for monitoring international normalized ratio (INR) level of a patient, comprising:
at a first device associated with the patient:
receiving, from an INR level measurement device at a first location, first INR data associated with the INR level of the patient;
receiving, from the patient, an input indicating warfarin dosage associated with the patient;
transmitting the first INR data and the warfarin dosage to a second device;
receiving a warfarin dosage recommendation from the second device, wherein the warfarin dosage recommendation is determined based on the transmitted first INR data and the transmitted warfarin dosage;
receiving second INR data associated with the INR level of the patient, wherein the second INR data is collected at a second location different the first location; and
presenting, on a display of the device associated with the patient, one or more user interfaces comprising:
a first user interface element indicating the warfarin dosage recommendation,
a second user interface element indicating a wellness of the INR level of the patient, the wellness of the INR level of the patient determined based on one or more of the first INR data received from the INR level measurement device at the first location and the second INR data collected at the second location, and
a third user interface element indicating a trend of the INR level of the patient, the trend determined based on a composite analysis of the first INR data received from the INR level measurement device at the first location and the second INR data collected at the second location.

2. The method of claim 1, further comprising receiving secondary data, wherein the warfarin dosage recommendation is determined further based on the secondary data.

3. The method of claim 2, wherein the secondary data comprises Virtual Health Record (VHR).

4. The method of claim 1, further comprising:
in accordance with a determination that the INR level of the patient is below a target INR level, presenting, on the display, a first user interface element indicating that the INR level is below the target INR level; and
in accordance with a determination that the INR level of the patient is not below the target INR level, presenting, on the display, a second user interface element indicating that the INR level is not below the target INR level.

5. The method of claim 1, further comprising presenting, on the display, a user interface element indicating a target INR level and a target time for reaching the target INR level, wherein the target INR level and the target time are determined based on one or more of the INR level of the patient, the warfarin dosage, and the warfarin dosage recommendation.

6. The method of claim 1, wherein the first INR data is received via a wireless connection.

7. The method of claim 1, wherein the warfarin dosage recommendation is received by a third device, different from the first device and the second device.

8. The method of claim 1, wherein the first INR data comprises clotting data of the patient, and the INR level of the patient is determined based on the clotting data of the patient.

9. The method of claim 1, further comprising inputting the first INR data and the warfarin dosage into a machine learning model.

10. The method of claim 1, wherein the warfarin dosage recommendation is determined based on an input provided to the second device in response to the transmission of the first INR data and the warfarin dosage.

11. The method of claim 1, wherein the warfarin dosage recommendation is determined by the second device.

12. The method of claim 1, wherein the third user interface element further indicates a correlation between the INR level and secondary data.

13. A system comprising:
an INR level measurement device at a first location;
a first device comprising a display,
a second device, and one or more processors, wherein the one or more processors are configured to perform any of the methods of claim 1.

14. A non-transitory computer-readable medium storing instructions that, when executed by one or more processors, cause the one or more processors to perform any of methods of claim 1.
